(19) United States
(12) Patent Application Publication
(10) Pub. No.: US 2004/0142340 A1
(43) Pub. Date: Jul. 22, 2004

(54) Detecting binding of mrna to an oligonucleotide array using rna dependent nucleic acid modifying enzymes

(76) Inventor: Peter Estibeiro, Midlothian (GB)

Correspondence Address: Fleshner & Kim, PO Box 221200, Chantilly, VA, 22151-1200, US

(21) Appl. No.: 10/469,931

(22) PCT Filed: Mar. 7, 2002
(86) PCT No.: PCT/GB02/01011

(30) Foreign Application Priority Data
Mar. 8, 2001 (GB) 0105790.0

(51) Int. Cl. C12Q 1/68; C12M 1/34

(52) U.S. Cl. 435/6; 435/287.2

(57) ABSTRACT

This invention relates to both a device and a method for detecting and binding of mRNA to oligonucleotides on an array. The oligonucleotides on the array have a reactive-OH group at their free 3′ end, and the binding of mRNA is detected using RNA dependent nucleic acid modifying enzymes.

This invention relates to a method of detecting the binding of mRNA to oligonucleotides on an array, wherein the binding of the mRNA is detected using RNA dependent nucleic acid modifying enzymes.

Antisense as a means of controlling gene expression for research or therapeutic purposes was first described in the 1970s. Since then, much effort has been put into understanding how antisense works and into developing methods to map effective antisense targets.

Antisense works by introducing a short synthetic nucleic acid, the antisense agent, that is complimentary to a target mRNA, into a cell. The antisense agent binds to its target mRNA and prevents translation by mechanisms thought to involve both tagging for degradation by endogenous nucleases and a physical hindrance of translocation or translation.

The design of antisense agents is complicated by the fact the mRNA has extremely complex secondary and tertiary structures. At least 90% of the nucleotide sequence of any given mRNA is involved in intra-molecular interactions within the secondary and tertiary structure of the molecule, and is thus unavailable to participate in inter-molecular interaction with an antisense agent. The key to the design of a successful antisense agent is to identify the limited regions of a potential mRNA target that are available for inter-molecular hybridisation. Antisense agents targeted specifically to these accessible regions have a high probability of binding to the target mRNA in vivo, and effectively knocking down the level of expression of its encoded product.

There are a number of in vitro experimental methods available in the prior art that can be used to effectively target antisense agents. Generally these experimental methods rely on using libraries of oligonucleotides to mediate cleavage of in vitro RNA transcripts by RNaseH, or to bind to the RNA and cause retardation of a labelled species during gel electrophoresis or to bind to the RNA on a array of separate elements, such that the signal from each element can be detected and correlated with ability to bind in that position.

Successful methods depend on knowing the sequence of the target mRNA and designing a library of overlapping oligonucleotides generally of up to twenty-five nucleotides in length. The sequence of the target mRNA is represented in the oligonucleotide library, such that the first oligonucleotide may be complimentary to positions one to fifteen on the target mRNA, the second will be complimentary to two to sixteen, and the third three to seventeen, etc.

Methods have been proposed in the prior art, whereby antisense agents can be designed using a device that is not dependent on advanced knowledge of the target sequence. Such a device is proposed to comprise an array of oligonucleotides immobilised on a glass or plastic support, such that all possible sequence combinations are represented by oligonucleotides of between four and eight nucleotide units. This type of device is disclosed in International Patent No WO98/15651 and also in U.S. Pat. No. 6,054,270. In this Patent and Patent Application each oligonucleotide is proposed to be physically separate on the array and after hybridisation of the target mRNA and washing off of unbound material, the signal is detected from the bound oligonucleotides. By knowing which sequences are present, and at which physically separate location on the array, the sequence of target mRNA that is accessible to inter-molecular hybridisation can be inferred. The inferred sequence is likely to be an effective target for antisense mediated gene knockdown.

Both WO98/15651 and U.S. Pat. No. 6,054,270 describe arrays comprising all possible four to eight base sequence combinations. While this is technologically feasible, with four base sequence combinations requiring 256 elements on an array to represent all sequence combinations, and eight base sequence combinations requiring 65,536 elements on an array to represent all sequence combinations, there are difficulties with using such short four to eight base oligonucleotides as the basis for an array. The difficulty with using short four to eight base oligonucleotides as the basis for an array to map RNA structure and define regions that are effective antisense targets, is that the interaction between the oligonucleotides on the array and the labelled transcript applied to it under hybridising conditions is very weak. Under normal washing conditions, the transcript is washed off and no signal is detected. This situation may be improved by spacing the short oligonucleotide array element away from the substrate of the array using a chemical spacer. Increasing salt concentration or decreasing temperature tends to increase non-specific background, but does not improve the signal. In Patent Application WO98/15651 it is demonstrated that a signal can be detected by hybridising RNA to four base oligonucleotides. However, under the conditions in the described protocol, there is a requirement that the RNA rather than the oligonucleotides is immobilised to the solid support and that the oligonucleotides are applied in solution to denatured RNA. Under these conditions, it is unlikely that the RNA would be folded into an authentic representation of its in vivo structure, and the method demonstrated would not map the structure of the RNA in a suitable manner to target antisense.

In short, the immobilised oligonucleotides will hybridise to full length RNA transcripts under conditions where the RNA concentration is sufficient to drive the hybridisation reaction in favour of forming duplexes. Under normal washing conditions, for example one M NaCl at between 4° C. and 30° C., the kinetic change associated with diluting away the applied RNA generally favours the melting apart of short duplexes, such that genuine interactions, signalling a region of the RNA may be accessible to antisense mediation knockdown, cannot be directly detected.

Typically, in order to get the consistency and conditions that will map RNA structure, where the RNA is folded in an authentic representation of its in vivo structure, it is necessary to use oligonucleotides of at least ten nucleotides in length. A fully degenerate array of oligonucleotides of ten nucleotides in length would comprise over one million elements and would be prohibitively expensive. Therefore, it can be seen that there is a requirement for a method that will enable detection of interactions between RNA and members of a combinatorial library of oligonucleotides of less than ten nucleotides in length.

It is a first object of this invention to provide a method of detecting hybridisation between a native in vitro RNA transcript and short oligonucleotides immobilised on a solid support.

It is a further object of this invention to provide a tool that will map accessible regions on any mRNA using a medium density oligonucleotide array consisting of up to 100,000 individual elements representing all possible combinations of a specific length or lengths of oligonucleotides.

It is also possible to use as the basis for an access mapping array a library of fully degenerate oligonucleotides of specific or varying length or lengths each of which is kept physically separate in solution such that the sequence of oligonucleotide in each solution is known. A subset or all of said oligonucleotides can be picked according to sequence and used to print an array complementary to the target RNA of interest. The selection and printing of said sub-set of oligonucleotides can be by computer controlled device.

It is a yet further object of this invention to provide a method for direct detection of duplexes.

It is a yet further still object of this invention to provide a method for indirect detection of duplexes formed between RNA and oligonucleotides.

According to the present invention, there is provided a device for determining structural parameters of native RNA by mapping RNA transcripts, comprising an array which has immobilised oligonucleotides represented on its surface support, wherein the oligonucleotides represented have a reactive —OH group at their free 3′-end, and a means for extending said —OH group that is dependent on a complementary base-pair interaction between specific immobilised oligonucleotides and the applied RNA.

Preferably the device further comprises a means of detecting said immobilised extension product.

Preferably, all possible combinations of a specific length or lengths of oligonucleotides are represented on the surface of the array.

More preferably, all possible combinations of oligonucleotides of six to eight or nine to fifteen nucleotides in length are represented at physically separate positions on the array.

Most preferably, all possible combinations of six to eight base oligonucleotides are represented at physically separate positions on the array.

Optionally, the array is made of plastic.

A further option is that the array is made of glass. This invention is not limited by the material of which the array is made.

Preferably the oligonucleotides are anchored to the array support by chemical modifications at 5′ end.

Preferably, the oligonucleotides are linked to the array using common linking techniques.

Optionally, the linking technique used to link oligonucleotides to the array is amino linking.

A further option for linking oligonucleotides to the array is to the use of biotin/streptavidin linking.

This invention is not limited by the linking technique used to link the oligonucleotides to the array.

Optionally the oligonucleotides are spaced away from the support by a chemical spacer.

Preferably, the chemical spacer is between 6 and 40 carbon equivalents in length.

The specific sequence of the oligonucleotides may be spaced away from the array by extending the 5′ end of the oligonucleotide using a plurality of nucleotides or nucleotide analogues. For example the six base sequence 5′CGGAAC3′ may be spaced from the array by making it 5′AAAAAAAAAAACGGAAC3′. The spacing nucleotides can be any natural or synthetic nucleotide or nucleotide analogue and can be a homopolymer or a heteropolymer. Nucleotide in this context is also taken to mean deoxynucleotide or any modified nucleotide or deoxynucleotide.

The nucleotide spacer may be used in conjunction with a chemical spacer

According to a second aspect of the present invention, there is provided a method for determining structural parameters of native RNA by detecting binding of said native RNA to an oligonucleotide array, as described in the first aspect, wherein:

(a) Native RNA is applied to the array and allowed to anneal to oligonucleotides complementary to accessible sequences within said native RNA; and

(b) an RNA dependent nucleic acid modifying enzyme is applied and allowed to react; and

(c) any unbound RNA or enzyme or other reaction components are washed off; and

(d) the modification(s) caused by the RNA dependent modifying enzyme is detected either directly or indirectly.

Preferably any or all of steps (a), (b), (c) and (d) are concurrent or sequential.

Preferably, the RNA dependent nucleic acid modifying enzyme is an RNA dependent DNA polymerase.

Most preferably the RNA dependent DNA polymerase is an enzyme with reverse transcriptase activity.

The enzyme with reverse transcriptase activity can be a modified reverse transcriptase lacking RNaseH activity.

Preferably a reaction buffer is used when the mRNA is applied to the array.

Most preferably the reaction buffer is compatible with enzyme activity and also with the maintenance of RNA secondary and tertiary structures and with duplex formation between the applied RNA and complementary oligonucleotide elements on the array.

Optionally a volume excluder is added to the reaction mix.

Optionally the volume excluder may be polyethylene glycol (PEG).

A further option is that the volume excluder may be dextran sulphate.

Optionally, labelled deoxynucleotide triphosphates (dNTPs) are applied to the array along with the RNA dependent nucleic acid modifying enzyme to allow for direct detection of duplexes.

Alternatively, labelled dideoxynucleotide triphosphates (ddNTPs) can be added along with the RNA dependent nucleic acid modifying enzyme to allow direct detection of duplexes.

Optionally, the labelled dNTPs or labelled ddNTPs can be detected by fluorescence, phosphorimaging or autoradiography.

Optionally, duplexes may be detected indirectly, by adding unlabelled ddNTPs to the array along with the RNA dependent nucleic acid modifying enzyme, washing off unreacted, excess ddNTPs, adding terminal transferase in an appropriate buffer with labelled dNTPs, wherein the immobilised oligonucleotides that do not become labelled are those that are complimentary to the accessible sequences of the previously applied target RNA.

In order to provide a better understanding of the invention, embodiments of the invention will now be described by way of example only.

In the first aspect of this invention, a device is provided for use in mapping RNA transcripts and determining regions that may be effective targets for antisense mediated gene knockdown. The device comprises an array which preferably has all possible combinations of a specific length or lengths of oligonucleotides represented with physically separate positions on the array. The oligonucleotides are anchored to the solid support by chemical modifications at their 5′ ends, such that they have a free reactive 3′ OH group.

The device is also provided with a means of extending said 3′ OH group that is dependent on a complementary base pair interaction between specific immobilised oligonucleotides and the applied RNA.

The chemical modifications that allow oligonucleotides to be immobilised to the surface of the array are usually in the form of the addition of linking groups. Generally amino linked oligonucleotides are used, although biotin/streptavidin linking is also a commonly used technique that could be applied to this invention, as could any other linking technology.

A sub-population of the oligonucleotides will hybridise to any applied RNA under conditions where the RNA is in sufficient concentration to drive the kinetics of a hybridisation reaction in favour of forming duplexes.

According to a second aspect of the present invention, the abovementioned array device can be used to detect the binding of unlabelled mRNA to a subset of oligonucleotides on an array.

A copy of the target mRNA is transcribed in vitro from a full length or partial cDNA clone under conditions in which the nascent RNA can fold in a manner that is an authentic representation of its in vivo structure. Once synthesised, the target mRNA is maintained under conditions that will maintain its authentic secondary and tertiary structure.

The target RNA is transcribed in vitro under conditions that allow authentic folding and applied to the array. The high concentration of applied RNA drives the kinetics of the hybridisation reaction between the RNA and those oligonucleotides on the array that are complimentary to the accessible regions of the RNA in the direction of duplex formation. Because of the orientation of the immobilised oligonucleotides, duplexes thus formed are substrates for RNA dependent DNA modifying enzymes, such as reverse transcriptase. In particular, enzymes such as AMV-reverse transcriptase or M-MuLV reverse transcriptase are appropriate. Reverse transcriptases engineered to stop their RNaseH activity may also be used, such as Expand RT (Roche) or M-MuLVRNaseH- (Promega). Generally, the RNA is applied to an array in a reaction buffer, which is compatible with enzymatic activity and also with the maintenance of RNA secondary and tertiary structures and supports duplex formation between the applied RNA and complementary oligonucleotide elements immobilised on the array. Typically, the reaction buffer may comprise 50 mM Tris-Cl, 5-10 mM MgCl₂, 25-50 mM KCl, 1-10 mM DTT at pH of approximately 8.5. A volume excluder such as polyethylene glycol (PEG) may be added to approximately 6% w/v or dextran sulphate to approximately 30% w/v. These volume excluders serve to increase the apparent concentration of reaction components and can have a favourable effect on the kinetics of duplex formation.

As the RNA/DNA hetroduplexes that are then formed are now substrates for RNA dependent DNA polymerases, such as reverse transcriptase, the reverse transcriptase can now be used to modify the free 3′ OH group of the hybridising oligonucleotide, and thus tag it for detection. By subsequently detecting modified oligonucleotides on the array, the sequences to which they were able to hybridise, and therefore the accessible regions of the applied RNA transcript can be inferred. The interaction is inferred either through direct or indirect interpretation of enzymatic modifications to the subset of oligonucleotides on the array.

Direct Detection of Duplexes

As discussed, the RNA in an appropriate buffer is applied to the array and allowed to hybridise to a subset of oligonucleotides which are complimentary to its accessible regions.

Reverse transcriptase is applied to the hybridised array and incubated for several minutes at between 4° C. and 65° C. Labelled deoxynucleotide triphosphates (dNTPs) are also added. Under these conditions, the reverse transcriptase will add labelled dNTPs to the free 3′ OH group of any hybridised oligonucleotides. The label may be either radioactive or fluorescent or may be an epitope, chemical tag or enzyme that can be subsequently detected by other standard means.

RNA, enzyme and any excess labelled dNTPs are washed off using an appropriate wash buffer, such as 100 mM NaCl, 0.1% SDS. Those oligonucleotides to which labelled dNTPs have been added can then be detected by the appropriate means, such as fluorescence, phosphorimaging or autoradiography. From the position on the array of the labelled oligonucleotides, the sequence is known and the complimentary accessible sequences on the applied RNA can be inferred.

Labelled dideoxynucleotide triphosphates (ddNTPs) can also be used for direct detection of duplexes, in which case the reverse transcriptase will add a single ddNTP to the free 3′ OH groups of hybridised oligonucleotides, as opposed to a dNTP as in the previous example. Detection is also carried out in the same way as when using dNTPs.

Indirect Detection of Duplexes

As before, RNA is applied to the array and reverse transcriptase is used to modify the three 3′ OH groups of hybridised oligonucleotides by the addition of a single unlabelled ddNTP.

RNA, enzyme and excess ddNTPs are washed off and the array is then equilibrated by washing twice in terminal transferase buffer. Terminal transferase and radio labelled or fluorescent or otherwise tagged dNTPs are applied to the array in terminal transferase buffer and incubated. Generally, incubation is at 37° C. for five minutes to thirty minutes. Terminal transferase can tail a free 3′ OH group by the addition of dNTPs, but this reaction is blocked by the addition of the unlabelled ddNTP as this results in the nucleic acid lacking a three 3′ OH group. The result of this sequence of reactions therefore, is that the oligonucleotides with the free 3′ OH group are those that did not initially hybridise to the applied RNA, and it is these oligonucleotides that are tailed when the terminal transferase and labelled dNTPs are applied. Oligonucleotides that did hybridise to the applied RNA are modified by the RNA dependent addition of a single ddNTP which is unlabelled, and these oligonucleotides can not be subsequently labelled by tailing with terminal transferase. Therefore, immobilised oligonucleotides that do not become labelled during the terminal transferase step are those that are complimentary to the accessible sequences of the applied target RNA.

The embodiments disclosed above are merely exemplary of the invention, which may be embodied in different forms. Therefore, the details disclosed herein are not to be interpreted as limiting, but merely taken as a basis for the Claims and teaching one skilled in the art as to the various uses of the present invention in any appropriate manner.

1. A device for determining structural parameters of native RNA by mapping RNA transcripts, comprising an array which has immobilised oligonucleotides represented on a surface support, wherein the oligonucleotides represented have a reactive-OH group at their free 3′ end, and a means of extending said 3′-OH group that is dependent on a complementary base-pair interaction between specific immobilised oligonucleotides and the applied RNA.

2. A device as described in claim 1, which further comprises a means of detecting said immobilised extension product.

3. A device for mapping RNA transcripts as described in claims 1 or 2, wherein any or all possible combinations of the specific length or lengths of oligonucleotides are represented on the surface of the array.

4. A device as described in any of the previous claims, wherein all possible combinations of oligonucleotides of 6 to 8 or 9 to 15 nucleotides in length are represented on the array.

5. A device as described in any of the previous claims, wherein the oligonucleotides are represented at physically separate positions on the array.

6. A device as described in any of the previous claims, wherein the oligonucleotides are anchored to the array support by chemical modifications at the 5′ end.

7. A device as described in any of the previous claims, wherein the oligonucleotides are linked to the array using common linking techniques.

8. A device as described in claim 7, wherein the linking technique used to link oligonucleotides to the array is amino linking.

9. A device as described in claim 8, wherein the linking technique used to link oligonucleotides to the array is biotin/streptavidin linking.

10. A device as described in any of the previous claims, wherein the oligonucleotides are spaced away from the support by a chemical spacer.

11. A device as described in claim 10, wherein the chemical spacer is between 6 and 40 carbon equivalents in length.

12. A device as described in claims 1 to 11, wherein the oligonucleotides may be spaced away from the support by extending the 5′ end of the oligonucleotide using a plurality of nucleotides or nucleotide analogues.

13. A device as described in claims 10 to 12, wherein the spacing nucleotides can be any natural or synthetic nucleotide or nucleotide analogue and can be a homopolymer or a heteropolymer.

14. A method for determining structural parameters of native RNA by detecting binding of said native RNA to an oligonucleotide array, wherein the following steps apply:
(a) Native RNA is applied to the array and allowed to anneal to oligonucleotides complementary to accessible sequences within said native RNA;
(b) an RNA dependent nucleic acid modifying enzyme is applied and allowed to react; and
(c) any unbound RNA or enzyme or other reaction components are washed off; and
(d) the modification(s) caused by the RNA dependent modifying enzyme is detected.

15. A method as described in claim 14, wherein any or all of steps (a) (b) (c) and (d) are concurrent or sequential.

16. A method as described in claims 14 or 15, wherein the modification or modifications caused by the RNA dependent modifying enzyme is detected directly.

17. A method as described in claims 14 or 15, wherein the modification or modifications caused by the RNA dependent modifying enzyme is detected indirectly.

18. A method as described in claims 14 to 17, wherein the RNA dependent nucleic acid modifying enzyme is a RNA dependent DNA polymerase.

19. A method as described in claim 18, wherein the RNA dependent DNA polymerase is an enzyme with reverse transcriptase activity.

20. A method as described in claim 19, wherein the enzyme with reverse transcriptase activity can be modified reverse transcriptase lacking RNaseH activity.

21. A method as described in any of claims 14 to 20, wherein a reaction buffer is used when the mRNA is applied to the array.

22. A method as described in claim 21, wherein the reaction buffer is compatible with enzyme activity and also with the maintenance of RNA secondary and tertiary structures and with the duplex formation between the applied RNA and the complementary oligonucleotide elements on the array.

23. A method as described in any of claims 14 to 22, wherein a volume excluder is added to the reaction mix.

24. A method as described in claim 23, wherein the volume excluder is polyethylene glycol (PEG).

25. A method as described in claim 23, wherein the volume excluder is dextran sulphate.

26. A method as described in any of claims 14 to 25, wherein labelled deoxynucleotide triphosphates (dNTPs) are applied to the array along with the RNA dependent nucleic acid modifying enzyme to allow for detection of duplexes.

27. A method as described in claims 14 to 25, wherein labelled dideoxynucleotide triphosphates (ddNTPs) can be added along with the RNA dependent nucleic acid modifying enzyme to allow the detection of duplexes.

28. A method as described in claims 26 or 27, wherein the labelled dNTPs or labelled ddNTPs can be detected by fluorescence, phosphorimaging or autoradiography.

29. A method as described in claims 14 to 25, wherein duplexes are detected by adding unlabelled ddNTPS to the array, along with the RNA dependent nucleic acid modifying enzyme, washing off unreacted excess ddNTPs, adding terminal transferase in an appropriate buffer with labelled dNTPs, so that the immobilised oligonucleotides that do not become labelled are those that are complementary to the accessible sequences of the previously applied target RNA.

30. A method as described in claims 14-29 wherein the array is a device as described in claims 1-13.
